(19) United States
(12) Patent Application Publication
(10) Pub. No.: US 2025/0387418 A1
(43) Pub. Date: Dec. 25, 2025

(54) USE OF CANNABINOIDS IN THE TREATMENT OF EPILEPSY

(71) Applicant: Jazz Pharmaceuticals Research UK Limited, Sittingbourne (GB)

(72) Inventors: Geoffrey GUY, Cambridge (GB); Stephen Wright, Cambridge (GB); Alice Mead, Cambridge (GB); Anne Comi, Baltimore, MD (US)

(21) Appl. No.: 19/309,408

(22) Filed: Aug. 25, 2025

Related U.S. Application Data
(63) Continuation of application No. 18/170,235, filed on Feb. 16, 2023, now abandoned.
Application No. 18/170,235 is a continuation of application No. 16/737,707, filed on Jan. 8, 2020, now abandoned.
Application No. 16/737,707 is a continuation of application No. 16/090,039, filed on Sep. 28, 2018, now Pat. No. 10,583,096.

(30) Foreign Application Priority Data
Mar. 31, 2016 (GB) 1605448.8

(51) Int. Cl. A61K 31/00; A61K 31/165; A61K 31/22; A61K 31/27; A61K 31/357; A61K 31/4015; A61K 31/55; A61K 31/5513; A61K 36/185; A61K 45/06; A61P 25/08

(52) U.S. Cl. CPC A61K 31/658; A61K 31/165; A61K 31/22; A61K 31/27; A61K 31/357; A61K 31/4015; A61K 31/55; A61K 31/5513; A61K 36/185; A61K 45/06; A61P 25/08

(57) ABSTRACT

The present invention relates to the use of cannabidiol (CBD) in the treatment of Sturge Weber syndrome. CBD ap-pears particularly effective in reducing all types of seizures and non-seizure symptoms in patients suffering with Sturge Weber syndrome. Preferably the CBD used is in the form of a highly purified extract of cannabis such that the CBD is present at greater than 98% of the total extract (w/w) and the other components of the extract are characterised. In particular the cannabinoid tetrahydrocan-nabinol (THC) has been substantially removed, to a level of not more than 0.15% (w/w) and the propyl analogue of CBD, cannabidivarin, (CBDV) is present in amounts of up to 1%. Alternatively, the CBD may be a synthetically produced CBD.

### FIELD OF THE INVENTION

The present invention relates to the use of cannabidiol (CBD) in the treatment of Sturge Weber syndrome. CBD appears particularly effective in reducing all types of seizures and non-seizure symptoms in patients suffering with Sturge Weber syndrome.

Preferably the CBD used is in the form of a highly purified extract of cannabis such that the CBD is present at greater than 98% of the total extract (w/w) and the other components of the extract are characterised. In particular the cannabinoid tetrahydrocannabinol (THC) has been substantially removed, to a level of not more than 0.15% (w/w) and the propyl analogue of CBD, cannabidivarin, (CBDV) is present in amounts of up to 1%. Alternatively, the CBD may be a synthetically produced CBD.

In use the CBD may be used concomitantly with one or more other anti-epileptic drugs (AED). Alternatively the CBD may be formulated for administration separately, sequentially or simultaneously with one or more AED or the combination may be provided in a single dosage form. Where the CBD is formulated for administration separately, sequentially or simultaneously it may be provided as a kit or together with instructions to administer the one or more components in the manner indicated. It may also be used as the sole medication, i.e. as a monotherapy.

### BACKGROUND TO THE INVENTION

Epilepsy occurs in approximately 1% of the population worldwide, (Thurman et al., 2011) of which 70% are able to adequately control their symptoms with the available existing anti-epileptic drugs (AED). However, 30% of this patient group, (Eadie et al., 2012), are unable to obtain seizure freedom using the AED that are available and as such are termed as suffering from intractable or “treatment-resistant epilepsy” (TRE).

Intractable or treatment-resistant epilepsy was defined in 2009 by the International League Against Epilepsy (ILAE) as “failure of adequate trials of two tolerated and appropriately chosen and used AED schedules (whether as monotherapies or in combination) to achieve sustained seizure freedom” (Kwan et al., 2009).

Individuals who develop epilepsy during the first few years of life are often difficult to treat and as such are often termed treatment-resistant. Children who undergo frequent seizures in childhood are often left with neurological damage which can cause cognitive, behavioral and motor delays.

Childhood epilepsy is a relatively common neurological disorder in children and young adults with a prevalence of approximately 700 per 100,000. This is twice the number of epileptic adults per population.

When a child or young adult presents with a seizure, investigations are normally undertaken in order to investigate the cause. Childhood epilepsy can be caused by many different syndromes and genetic mutations and as such diagnosis for these children may take some time.

The main symptom of epilepsy is repeated seizures. In order to determine the type of epilepsy or the epileptic syndrome that a patient is suffering from, an investigation into the type of seizures that the patient is experiencing is undertaken. Clinical observations and electroencephalography (EEG) tests are conducted and the type(s) of seizures are classified according to the ILAE classification described below and in FIG. 1.

The International classification of seizure types proposed by the ILAE was adopted in 1981 and a revised proposal was published by the ILAE in 2010 and has not yet superseded the 1981 classification. FIG. 1 is adapted from the 2010 proposal for revised terminology and includes the proposed changes to replace the terminology of partial with focal. In addition the term “simple partial seizure” has been replaced by the term “focal seizure where awareness/responsiveness is not impaired” and the term “complex partial seizure” has been replaced by the term “focal seizure where awareness/consciousness is impaired”.

From FIG. 1 it can be seen that Generalised seizures, where the seizure arises within and rapidly engages bilaterally distributed networks, can be split into six subtypes: Tonic-Clonic (grand mal) seizures; Absence (petit mal) Seizures; Clonic Seizures; Tonic Seizures; Atonic Seizures and Myoclonic Seizures.

Focal (partial) seizures where the seizure originates within networks limited to only one hemisphere, are also split into sub-categories. Here the seizure is characterized according to one or more features of the seizure, including aura, motor, autonomic and awareness/responsiveness. Where a seizure begins as a localized seizure and rapidly evolves to be distributed within bilateral networks this seizure is known as a Bilateral convulsive seizure, which is the proposed terminology to replace Secondary Generalized Seizures (generalized seizures that have evolved from focal seizures and no longer remain localized).

Focal seizures where the subject's awareness/responsiveness is altered are referred to as focal seizures with impairment and focal seizures where the awareness or responsiveness of the subject is not impaired are referred to as focal seizures without impairment.

Epileptic syndromes often present with many different types of seizure and identifying the types of seizure that a patient is suffering from is important as many of the standard AED's are targeted to treat or are only effective against a given seizure type/subtype.

One such childhood epilepsy is Sturge Weber syndrome (SWS). SWS is a congenital, non-familial disorder of unknown incidence. It is caused by a somatic genetic mutation in the gene GNAQ. It is characterized by a congenital facial birthmark and neurological abnormalities. Other symptoms associated with SWS can include eye and internal organ irregularities.

The clearest indication of SWS is a facial birthmark or “Port Wine Stain” which is present from birth. This typically involves at least one upper eyelid and the forehead.

Neurological problems are caused by the development of excessive blood vessel growth on the surface of the brain. These are located typically on the back region of the brain on the same side as the port wine birthmark. These growths create abnormal conditions for brain function in the region.

Epilepsy is the most common early problem, (in around 80% of children with SWS), often starting by one year of age. The convulsions usually appear on the opposite side of the body from the port wine stain and vary in severity. A weakening or loss of the use of one side of the body may develop opposite to the port wine stain. Developmental delay of motor and cognitive skills may also occur to varying degrees.

Other problems such as visual field defects, glaucoma and headaches often occur in addition to neurological problems.

The anticonvulsant medications used to treat seizures in SWS include carbamazepine; valproate; acetazolamide; diazepam; phenytoin; felbamate; tiagabine; levetiracetam; clonazepam; lamotrigine; primidone; gabapentin; phenobarbital; ethosuximide and zonisamide and topiramate.

Management of the non-seizure symptoms in SWS are also required.

Common AED defined by their mechanisms of action are described in the following tables:

TABLE 1
Examples of narrow spectrum AED

| Narrow- | | |
|---|---|---|
| spectrum AED | Mechanism | Indication |
| Phenytoin | Sodium channel | Complex partial |
| | | Tonic-clonic |
| Phenobarbital | GABA/Calcium | Partial seizures |
| | channel | Tonic-clonic |
| Carbamazepine | Sodium channel | Partial seizures |
| | | Tonic-clonic |
| | | Mixed seizures |
| Oxcarbazepine | Sodium channel | Partial seizures |
| | | Tonic-clonic |
| | | Mixed seizures |
| Gabapentin | Calcium channel | Partial seizures |
| | | Mixed seizures |
| Pregabalin | Calcium channel | Adjunct therapy for partial |
| | | seizures with or without |
| | | secondary generalisation |
| Lacosamide | Sodium channel | Adjunct therapy for partial |
| | | seizures |
| Vigabatrin | GABA | Secondarily generalized |
| | | tonic-clonic seizures |
| | | Partial seizures |
| | | Infantile spasms due to |
| | | West syndrome |

TABLE 2
Examples of broad spectrum AED

| Broad- | | |
|---|---|---|
| spectrum AED | Mechanism | Indication |
| Valproic acid | GABA/Sodium | First-line treatment for |
| | channel | tonic-clonic seizures, |
| | | absence seizures and |
| | | myoclonic seizures |
| | | Second-line treatment |
| | | for partial seizures |
| | | and infantile spasms. |
| | | Intravenous use in |
| | | status epilepticus |
| Lamotrigine | Sodium channel | Partial seizures |
| | | Tonic-clonic |
| | | Seizures associated with |
| | | Lennox-Gastaut syndrome |
| Ethosuximide | Calcium channel | Absence seizures |
| Topiramate | GABA/Sodium channel | Seizures associated with |
| | | Lennox-Gastaut syndrome |
| Zonisamide | GABA/Calcium/Sodium | Adjunctive therapy in |
| | channel | adults with partial- |
| | | onset seizures |
| | | Infantile spasm |
| | | Mixed seizure |
| | | Lennox-Gastaut syndrome |
| | | Myoclonic |
| | | Generalised tonic-clonic |
| | | seizure |
| Levetiracetam | Calcium channel | Partial seizures |
| | | Adjunctive therapy for |
| | | partial, myoclonic and |
| | | tonic-clonic seizures |
| Clonazepam | GABA | Typical and atypical |
| | | absences |
| | | Infantile myoclonic |
| | | Myoclonic seizures |
| | | Akinetic seizures |
| Rufinamide | Sodium channel | Adjunctive treatment of |
| | | partial seizures |
| | | associated with Lennox- |
| | | Gastaut syndrome |

TABLE 3
Examples of AED used specifically in childhood epilepsy

| AED | Mechanism | Indication |
|---|---|---|
| Clobazam | GABA | Adjunctive therapy in complex |
| | | partial seizures |
| | | Status epilepticus |
| | | Myoclonic |
| | | Myoclonic-absent |
| | | Simple partial |
| | | Complex partial |
| | | Absence seizures |
| | | Lennox-Gastaut syndrome |
| Stiripentol | GABA | Severe myoclonic epilepsy in |
| | | infancy (Dravet syndrome) |

Over the past forty years there have been a number of animal studies on the use of the non-psychoactive cannabinoid cannabidiol (CBD) to treat seizures. For example, Consroe et al., (1982) determined that CBD was able to prevent seizures in mice after administration of pro-convulsant drugs or an electric current.

Studies in epileptic adults have also occurred in the past forty years with CBD. Cunha et al. reported that administration of CBD to eight adult patients with secondary generalized epilepsy resulted in a marked reduction of seizures in 4 of the patients (Cunha et al., 1980).

A study in 1978 provided 200 mg/day of pure CBD to four adult patients, two of the four patients became seizure free, whereas in the remainder seizure frequency was unchanged (Mechoulam and Carlini, 1978).

In contrast to the studies described above, an open label study reported that 200 mg/day of pure CBD was ineffective in controlling seizures in twelve institutionalized adult patients (Ames and Cridland, 1986).

Based on the fact that chronologically the last study to look at the effectiveness of CBD in patients with epilepsy proved that CBD was unable to control seizures, there would be no expectation that CBD might be useful as an anti-convulsant agent.

In the past forty years of research there have been over thirty drugs approved for the treatment of epilepsy none of which are cannabinoids. Indeed, there appears to have been a prejudice against cannabinoids, possibly due to the scheduled nature of these compounds and/or the fact that THC, which is a known psychoactive, has been ascribed as a pro-convulsant (Consroe et al., 1977).

The patent applications GB 2,487,712 describes the use of CBD with anti-epileptic drugs and WO 2015/193667 describes the use of CBD in the treatment of treatment resistant epilepsy, in particular patients with FIRES are shown to benefit particularly from the treatment.

A paper published recently suggested that cannabidiol-enriched cannabis may be efficacious in the treatment of epilepsy. Porter and Jacobson (2013) report on a parent survey conducted via a Facebook group which explored the use of cannabis which was enriched with CBD in children with treatment-resistant epilepsy. It was found that sixteen of the 19 parents surveyed reported an improvement in their child's epilepsy. The children surveyed for this paper were all taking cannabis that was purported to contain CBD in a high concentration although the amount of CBD present and the other constituents including THC were not known for many of the cases. Indeed, whilst CBD levels ranged from 0.5 to 28.6 mg/kg/day (in those extracts tested), THC levels as high as 0.8 mg/kg/day were reported.

A paper by Press et al. (2015) describes a review of 75 children and adolescents provided with oral cannabis extract. The responder rate for patients with Lennox-Gastaut syndrome was very high at 88.9%, whereas the rate for other childhood epilepsy syndromes such as Doose syndrome and Dravet syndrome were much lower or showed no improvement at all.

Providing children with TRE with a cannabis extract that comprises THC, which has been described as a pro-convulsant (Consroe et al., 1977), at a potentially psychoactive dose of 0.8 mg/kg/day, is a concern and as such there is a need to determine whether CBD is in fact efficacious.

More recently in March 2016, GW Pharmaceuticals announced positive results in a Phase 3 study of CBD in the treatment of Dravet syndrome.

To date there have been no trials of CBD in children and young adults with Sturge Weber syndrome.

### BRIEF SUMMARY OF THE DISCLOSURE

In accordance with a first aspect of the present invention there is provided Cannabidiol (CBD) for use in the treatment of Sturge Weber syndrome.

Preferably the CBD is used in the treatment of seizures, both non-convulsive and convulsive in Sturge Weber syndrome.

In a further embodiment the CBD is used in the treatment of non-seizure symptoms in Sturge Weber syndrome.

Preferably the non-seizure symptoms are one or more of mood, behaviour, cognitive function and general quality of life.

In a further embodiment the CBD is for use in combination with one or more concomitant anti-epileptic drugs (AED).

In a further embodiment the CBD is present as a highly purified extract of cannabis which comprises at least 98% (w/w) CBD. Preferably the extract comprises less than 0.15% THC. More preferably the extract further comprises up to 1% CBDV. More preferably still the extract further comprises between 0.1 and 1.0% CBDV.

In an alternative embodiment the CBD is present as a synthetic compound.

In a further embodiment of the invention the one or more AED is selected from the group consisting of: carbamezapine, clobazam, clonazepam, clonidine, clorazepate, desmethylclobazam, diazepam, ethosuximide, felbamate, ketogenic diet, lacosamide, lamotrigine, levetiracetam, lorazepam, midazolam, N-desmethylclobazam, nordiazepam, oxycarbamezapine, perampanel, phenobarbital, phenytoin, pregabalin, rufinamide, stiripentol, topiramate, trazodone, vagus nerve stimulation, valproic acid, vigabatrin, and zonisamide.

Preferably the CBD is used in combination with at least two or more AED and may be particularly beneficial when the patient is taking valproate.

Preferably the number of different anti-epileptic drugs that are used in combination with the CBD is reduced. Alternatively the dose of anti-epileptic drugs that are used in combination with the CBD is reduced.

There are many side effects associated with the commonly used AED which include dizziness, blurred vision, nausea, respiratory system depression, tiredness, headaches, and other motor side effects on the central nervous system. These side effects are particularly common as higher doses or combinations of numerous AED are used. As such there is a need for an alternative medication that is able to reduce the numbers of seizures whilst at the same time exhibiting a safe side effect profile.

Preferably the dose of CBD is greater than 5 mg/kg/day. Thus for a 15 kg patient a dose of greater than 75 mg of CBD per day would be provided. Doses greater than 5 mg/kg/day such as greater than 10/mg/kg/day, greater than 15 mg/kg/day, greater than 20 mg/kg/day and greater than 25 mg/kg/day are also envisaged to be effective.

Preferably the CBD is for use in the treatment of children and young adults with Sturge Weber syndrome.

In accordance with a second aspect of the present invention there is provided a method of treating Sturge Weber syndrome comprising administering cannabidiol (CBD) to a subject.

Preferably the subject is a human, more preferably a child or young adult.

Preferably the CBD is used in the treatment of seizures, both non-convulsive and convulsive in Sturge Weber syndrome.

In a further embodiment the CBD is used in the treatment of non-seizure symptoms in Sturge Weber syndrome.

Preferably the non-seizure symptoms are one or more of mood, behaviour, cognitive function and general quality of life.

### Definitions

Definitions of some of the terms used to describe the invention are detailed below:

The cannabinoids described in the present application are listed below along with their standard abbreviations.

TABLE 4
Cannabinoids and their abbreviations

| | | |
|---|---|---|
| CBD | Cannabidiol | 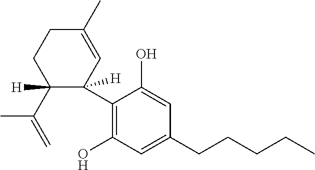 [SMILES: C=C(C)[C@@H]1CCC(C)=C[C@H]1c1c(O)cc(CCCCC)cc1O] |
| CBDA | Cannabidiolic acid | 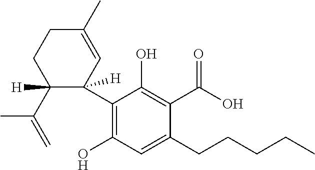 [SMILES: C=C(C)[C@@H]1CCC(C)=C[C@H]1c1c(O)cc(CCCCC)c(C(=O)O)c1O] |
| CBDV | Cannabidivarin | 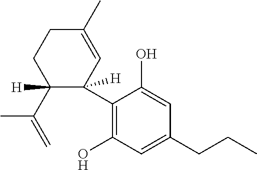 [SMILES: C=C(C)[C@@H]1CCC(C)=C[C@H]1c1c(O)cc(CCC)cc1O] |
| CBDVA | Cannabidivarinic acid | 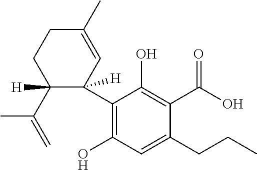 [SMILES: C=C(C)[C@@H]1CCC(C)=C[C@H]1c1c(O)cc(CCC)c(C(=O)O)c1O] |
| THC | Tetrahydrocannabinol | 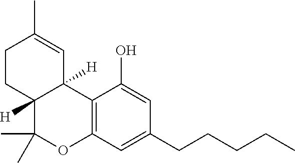 [SMILES: CCCCCc1cc(O)c2c(c1)OC(C)(C)[C@@H]1CCC(C)=C[C@@H]21] |

The table above is not exhaustive and merely details the cannabinoids which are identified in the present application for reference. So far over 60 different cannabinoids have been identified and these cannabinoids can be split into different groups as follows:

Phytocannabinoids; Endocannabinoids and Synthetic cannabinoids (which may be novel cannabinoids or synthetically produced phytocannabinoids or endocannabinoids).

“Phytocannabinoids” are cannabinoids that originate from nature and can be found in the cannabis plant. The phytocannabinoids can be isolated from plants to produce a highly purified extract or can be reproduced synthetically.

“Highly purified cannabinoid extracts” are defined as cannabinoids that have been extracted from the cannabis plant and purified to the extent that other cannabinoids and non-cannabinoid components that are co-extracted with the cannabinoids have been substantially removed, such that the highly purified cannabinoid is greater than or equal to 98% (w/w) pure.

“Synthetic cannabinoids” are compounds that have a cannabinoid or cannabinoid-like structure and are manufactured using chemical means rather than by the plant.

Phytocannabinoids can be obtained as either the neutral (decarboxylated form) or the carboxylic acid form depending on the method used to extract the cannabinoids. For example it is known that heating the carboxylic acid form will cause most of the carboxylic acid form to decarboxylate into the neutral form.

“Treatment-resistant epilepsy” (TRE) or “intractable epilepsy” is defined as per the ILAE guidance of 2009 as epilepsy that is not adequately controlled by trials of one or more AED.

“Childhood epilepsy” refers to the many different syndromes and genetic mutations that can occur to cause epilepsy in childhood. Examples of some of these are as follows: Dravet Syndrome; Myoclonic-Absence Epilepsy; Lennox-Gastaut syndrome; Generalized Epilepsy of unknown origin; CDKL5 mutation; Aicardi syndrome; bilateral polymicrogyria; Dup15q; SNAP25; and febrile infection related epilepsy syndrome (FIRES); benign rolandic epilepsy; juvenile myoclonic epilepsy; Sturge Weber Syndrome (SWS); infantile spasm (West syndrome); and Landau-Kleffner syndrome. The list above is non-exhaustive as many different childhood epilepsies exist.

“Focal Seizures” are defined as seizures which originate within networks limited to only one hemisphere. What happens during the seizure depends on where in the brain the seizure happens and what that part of the brain normally does.

“Focal seizure where awareness/consciousness are impaired” has replaced the term “complex partial seizure”. These seizures usually start in a small area of the temporal lobe or frontal lobe of the brain and involve other areas of the brain within the same hemisphere that affect alertness and awareness. Most subjects experience automatisms during a focal seizure with impaired consciousness.

“Percentage decrease in seizure frequency” is defined as the number of seizures at week 14 minus the number of seizures at baseline divided by the number of seizures at baseline multiplied by 100. In patients who are poor responders to existing AED any improvement in response particularly where the improvement is without side effects such as motor side effects on the central nervous system is highly desirable.

### BRIEF DESCRIPTION OF THE DRAWINGS

FIG. 1 shows the ILAE proposal for revised terminology for organization of seizures and epilepsies.

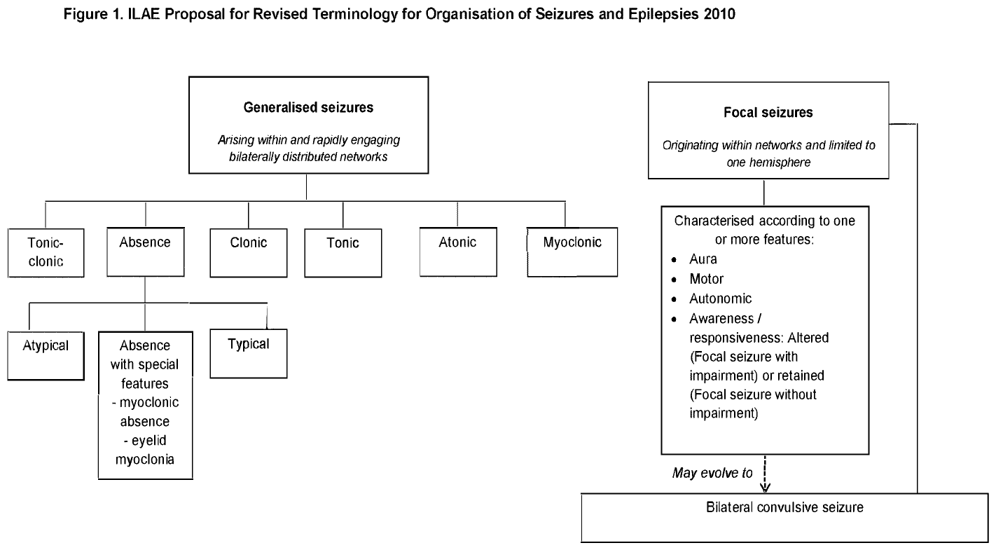

### DETAILED DESCRIPTION

### Preparation of Highly Purified CBD Extract

The following describes the production of the highly-purified (>98% w/w) cannabidiol extract which has a known and constant composition which was used for the expanded access trials described in the Examples below.

In summary the drug substance used in the trials is a liquid carbon dioxide extract of high-CBD containing chemotypes of Cannabis sativa L. which had been further purified by a solvent crystallization method to yield CBD. The crystallisation process specifically removes other cannabinoids and plant components to yield greater than 98% CBD.

The Cannabis sativa L. plants are grown, harvested, and processed to produce a botanical extract (intermediate) and then purified by crystallization to yield the CBD (drug substance).

The plant starting material is referred to as Botanical Raw Material (BRM); the botanical extract is the intermediate; and the active pharmaceutical ingredient (API) is CBD, the drug substance.

Both the botanical starting material and the botanical extract are controlled by specifications. The drug substance specification is described in Table 5 below.

TABLE 5
CBD Specification

| Test | Test Method | Limits |
|---|---|---|
| Appearance | Visual | Off-white/pale yellow crystals |
| Identification A | HPLC-UV | Retention time of major peak |
| | | corresponds to certified |
| | | CBD Reference Standard |
| Identification B | GC-FID/MS | Retention time and mass |
| | | spectrum of major peak |
| | | corresponds to certified |
| | | CBD Reference Standard |
| Identification C | FT-IR | Conforms to reference |
| | | spectrum for certified |
| | | CBD Reference Standard |
| Identification D | Melting Point | 65-67° C. |
| Identification E | Specific | Conforms with certified CBD |
| | Optical | Reference Standard; −110° |
| | Rotation | to −140° (in 95% ethanol) |
| Total Purity | Calculation | ≥98.0% |
| Chromatographic | HPLC-UV | ≥98.0% |
| Purity 1 | | |
| Chromatographic | GC-FID/MS | ≥98.0% |
| Purity 2 | | |
| Other | HPLC-UV | |
| Cannabinoids: | | |
| CBDA | | NMT 0.15% w/w |
| CBDV | | NMT 1.0% w/w |
| Δ⁹ THC | | NMT 0.15% w/w |
| CBD-C4 | | NMT 0.5% w/w |
| Residual Solvents: | GC | |
| Alkane | | NMT 0.5% w/w |
| Ethanol | | NMT 0.5% w/w |
| Residual Water | Karl Fischer | NMT 1.0% w/w |

NMT—Not more than

The purity of the CBD drug substance achieved is greater than 98%. The other cannabinoids which may occur in the extract are: CBDA, CBDV, CBD-C4 and THC.

The CBDV may be present in the drug substance at an amount of between 0.1 and 1%.

Distinct chemotypes of Cannabis sativa L. plant have been produced to maximize the output of the specific chemical constituents, the cannabinoids. One type of plant produces predominantly CBD. Only the (−)-trans isomer occurs naturally. Furthermore during purification the stereochemistry of CBD is not affected.

### Production of the Intermediate

An overview of the steps to produce a botanical extract, the intermediate, are as follows:
1. Growing
2. Decarboxylation
3. Extraction No. 1—using liquid CO2
4. Extraction No. 2—‘winterization’ using ethanol
5. Filtration
6. Evaporation

High CBD chemovars were grown, harvested and dried and stored in a dry room until required. The botanical raw material (BRM) was finely chopped using an Apex mill fitted with a 1 mm screen. The milled BRM was stored in a freezer for up to 3 months prior to extraction.

Decarboxylation of CBDA to CBD was carried out using a large Heraeus tray oven. The decarboxylation batch size in the Heraeus is approximately 15 Kg. Trays were placed in the oven and heated to 105° C.; the BRM took 96.25 minutes to reach 105° C. Held at 105° C. for 15 Minutes. Oven then set to 150° C.; the BRM took 75.7 minutes to reach 150° C.; BRM held at 150° C. for 130 Minutes. Total time in the oven was 380 Minutes, including 45 minutes cooling and 15 Minutes venting.

Extraction No 1 was performed using liquid CO2 at 60 bar/10° C. to produce botanical drug substance (BDS).

The crude CBD BDS was winterised in Extraction No 2 under standard conditions (2 volumes of ethanol at minus 20° C. for around 50 hours). The precipitated waxes were removed by filtration and the solvent evaporated using the rotary evaporator (water bath up to 60° C.) to yield the BDS, which was then used for crystallisation to produce the test material.

### Production of the Drug Substance

The manufacturing steps to produce the drug substance from the intermediate botanical extract are as follows:
1. Crystallization using C5-C12 straight chain or branched alkane
2. Filtration
3. Optional recrystallization from C5-C12 straight chain or branched alkane
4. Vacuum drying

Intermediate botanical extract (12 kg) produced using the methodology above was dispersed in C5-C12 straight chain or branched alkane (9000 ml, 0.75 vols) in a 30 litre stainless steel vessel.

[The mixture was manually agitated to break up any lumps and the sealed container then placed in a freezer for approximately 48 hours.

The crystals were isolated by vacuum filtration, washed with aliquots of cold C5-C12 straight chain or branched alkane (total 12000 ml), and dried under a vacuum of <10 mb at a temperature of 60° C. until dry before submitting the drug substance for analysis.

The dried product was stored in a freezer at minus 20° C. in a pharmaceutical grade stainless steel container, with FDA food grade approved silicone seal and clamps.

### Production of the Drug Product

The drug product is presented as an oral solution. The oral solution presentation contains 25 mg/ml or 100 mg/ml CBD, with the excipients sesame oil, ethanol, sucralose and flavouring. Two product strengths are available to allow dose titration across a wide dose range.

The 25 mg/ml solution is appropriate at lower doses and the 100 mg/ml solution at higher doses.

The drug product formulation is as described in Table 6 below:

TABLE 6
Drug Product specification

| | | | | Reference |
|---|---|---|---|---|
| | Qualitative | | | to Quality |
| Component | Composition | | Function | Standard |
| Cannabidiol (CBD) | 25 mg/ml or | | Active | In-house |
| | 100 mg/ml | | | |
| Anhydrous ethanol | 79.0 | mg/ml* | Excipient | Ph. Eur. |
| Sucralose | 0.5 | mg/ml | Sweetener | In-house |
| Strawberry flavouring | 0.2 | mg/ml | Flavouring | In-house |

| | | | |
|---|---|---|---|
| Sesame oil | q.s to 1.0 ml | Excipient | Ph. Eur. |

The drug substance, CBD is insoluble in water. Sesame oil was selected as an excipient to solubilize the drug substance.

A sweetener and fruit flavouring are required to improve palatability of the sesame oil solution.

Ethanol was required to solubilize the sweetener and the flavouring.

The composition can be substantially equivalent, by which is meant the functional ingredients can vary from the qualitative composition specified in Table 6 by an amount of up to 10%.

Example 1 below describes the use of a highly purified cannabis extract comprising cannabidiol (CBD). Cannabidiol is the most abundant non-psychoactive cannabinoid in the selected chemovar. Previous studies in animals have demonstrated that CBD has anticonvulsant efficacy in multiple species and models.

Example 1 describes data produced in an expanded access treatment program in children with SWS.

### Example 1: Efficacy of Cannabidiol in Reducing Seizures and Other Symptoms in Children and Young Adults with Sturge Weber Syndrome

### Materials and Methods

Four subjects with SWS brain involvement and refractory epilepsy were enrolled in an expanded access compassionate use program for CBD. These subjects were treated with a highly purified extract of cannabidiol (CBD) obtained from a cannabis plant. Frequency of seizures was recorded at each visit, as were reported quality of life changes, including mood, behaviour, and cognitive function.

Data were compared in the 56-day pre-treatment period, the 56-day period after starting maintenance dose (Week 14), and at most recent visit.

The participants in the study were taking at the time of entry into the study between one and four concomitant AEDs.

Patient 1 was taking leviteracetam alone. Patient 2 was taking leviteracetam, valproate, felbamate and clobazam. Patient 3 was taking valproate and topiramate. Patient 4 was taking oxycarbamazepine and lacosamide.

CBD was administered at a target dose of 15-25 mg/kg/day.

All patients entered a baseline period of 14 weeks when parents/caregivers kept prospective seizure diaries, noting all countable seizure types.

The patients then received a highly purified CBD extract (greater than 98% CBD w/w) in sesame oil, of known and constant composition, at a dose of 5 mg/kg/day in addition to their baseline anti-epileptic drug (AED) regimen.

The daily dose was gradually increased by 2 to 5 mg/kg increments up to a maximum dose of 25 mg/kg/day.

Patients were seen at regular intervals of 2-4 weeks. Laboratory testing for hematologic, liver, kidney function and concomitant AED levels was performed at baseline, and after every 4 weeks of CBD therapy.

### Results

At Week 14, all 4 patients had a reduction in the number of seizures of between 10 and 90%.

A summary of the improvement in total seizure frequency, based on 14 weeks of treatment are summarized in Table 7 below.

TABLE 7
Summary of total seizure frequency after 14 weeks of treatment

| | Baseline | Week 14 | Decrease in |
|---|---|---|---|
| Patient | (number of | (number of | seizure |
| number | seizures) | seizures) | frequency (%) |
| 1 | 67 | 60 | 10 |
| 2 | 6 | 1 | 83 |
| 3 | 10 | 1 | 90 |
| 4 | 6 | 4 | 33 |

Table 7 shows that after 14 weeks of therapy, two patients experienced a dramatic reduction in seizure frequency at 83 and 90% reduction. Both of the other patients with SWS experienced a decrease in seizures albeit to a lesser extent. These data infer that the CBD is very effective at reducing seizure in this type of epilepsy syndrome.

In addition three of the four patients (patients 2, 3 and 4) reported improved quality of life, whereas one (patient 1) remained unchanged. These included improvements in mood and behaviour in two subjects (patient 2 and 3) and improvements in cognitive function in three subjects (patients 2, 3 and 4).

### CONCLUSIONS

These data indicate that CBD is effective in the treatment of SWS.

Particular benefits include reducing in seizure frequency in a high proportion of patients with SWS that do not respond well to existing AED.

Indeed it is interesting to note that the two patients receiving valproate (patient numbers 2 and 3) obtained the greatest benefit and the patient with the least benefit was only taking one AED whilst the remainder of the patients were taking at least two concomitant AED. It was surprising that in this group of patients which are treatment-resistant such a high number were able to gain an effect. The fact that three quarters of the patients benefitted from a reduction in the number of seizures that they suffered from was remarkable. This might be a consequence of a synergy with valproate or another AED or the fact that more than one AED was used concomitantly with the CBD.

It is also surprising that the patients a high proportion of the patients additionally experienced improvements in other areas of their disease such as improvements in mood, behaviour, cognitive function and general quality of life.

### REFERENCES

Ames F R and Cridland S (1986). “Anticonvulsant effects of cannabidiol.” S Afr Med J 69:14.
Consroe P, Martin P, Eisenstein D. (1977). “Anticonvulsant drug antagonism of delta-9-tetrahydrocannabinol induced seizures in rabbits.” Res Commun Chem Pathol Pharmacol. 16:1-13
Consroe P, Benedicto M A, Leite J R, Carlini E A, Mechoulam R. (1982). “Effects of cannabidiol on behavioural seizures caused by convulsant drugs or current in mice.” Eur J Pharmaco. 83:293-8
Cunha J M, Carlini E A, Pereira A E, Ramos O L, Pimental C, Gagliardi R et al. (1980). “Chronic administration of cannabidiol to healthy volunteers and epileptic patient.” Pharmacology. 21:175-85
Dravet C. The core Dravet syndrome phenotype. Epilepsia. 2011 April; 52 Suppl 2:3-9.
Eadie, M J (December 2012). “Shortcomings in the current treatment of epilepsy.” Expert Review of Neurotherapeutics 12 (12): 1419-27.
Kwan P, Arzimanoglou A, Berg A T, Brodie M J, Hauser W A, Mathern G, Moshe S L, Perucca E, Wiebe S, French J. (2009) “Definition of drug resistant epilepsy: Consensus proposal by the ad hoc Task Force of the ILAE Commission on Therapeutic Strategies.” Epilepsia.
Mechoulam R and Carlini E A (1978). “Toward drugs derived from cannabis.” Die naturwissenschaften 65:174-9.
Porter B E, Jacobson C (December 2013). “Report of a parent survey of cannabidiol-enriched cannabis use in paediatric treatment resistant epilepsy” Epilepsy Behaviour. 29(3) 574-7
Press C A, Knupp K G and Chapman K E (2015) “Parental reporting of response to oral cannabis extracts for treatment of refractory epilepsy”. Epilepsy and Behaviour. 45. 49-52.
Thurman, D J; Beghi, E; Begley, C E; Berg, A T; Buchhalter, J R; Ding, D; Hesdorffer, D C;
Hauser, W A; Kazis, L; Kobau, R; Kroner, B; Labiner, D; Liow, K; Logroscino, G; Medina, M T; Newton, C R; Parko, K; Paschal, A; Preux, P M; Sander, J W; Selassie, A; Theodore, W;
Tomson, T; Wiebe, S; ILAE Commission on, Epidemiology (September 2011). “Standards for epidemiologic studies and surveillance of epilepsy.” Epilepsia. 52 Suppl 7:2-26

1. A method of treating Sturge Weber syndrome in a subject, comprising administering to the subject a therapeutically effective amount of cannabidiol (CBD) drug substance, wherein the CBD drug substance comprises at least 98% (w/w) CBD.

2. The method of claim 1, wherein the treating comprises treating seizures in a subject having Sturge Weber syndrome.

3. The method of claim 1, wherein the treating comprises treating non-seizure symptoms in a subject having Sturge Weber syndrome.

4. The method of claim 3, wherein the non-seizure symptoms are one or more of mood, behavior, cognitive function and general quality of life.

5. The method of claim 1, wherein the drug substance comprises less than 0.15% tetrahydrocannabinol (THC).

6. The method of claim 1, wherein the drug substance further comprises up to 1% cannabidivarin (CBDV).

7. The method of claim 1, wherein the drug substance further comprises between 0.1 and 1.0% CBDV.

8. The method of claim 1, wherein the CBD is present as a synthetic compound.

9. The method of claim 1, wherein the dose of CBD is about 10 mg/kg/day.

10. The method of claim 1, wherein the subject is a child or young adult.

11. The method of claim 1, wherein the dose of CBD ranges from about 5 mg/kg/day to about 25 mg/kg/day.

12. The method of claim 1, wherein the dose of CBD is about 20 mg/kg/day.

13. The method of claim 1, wherein the dose of CBD is about 25 mg/kg/day.

14. The method of claim 1, wherein the CBD drug substance is a highly purified extract of cannabis.

15. The method of claim 2, wherein the dose of CBD ranges from about 5 mg/kg/day to about 25 mg/kg/day.

16. The method of claim 2, wherein the dose of CBD is about 10 mg/kg/day.

17. The method of claim 2, wherein the dose of CBD is about 20 mg/kg/day.

18. The method of claim 2, wherein the dose of CBD is about 25 mg/kg/day.

19. The method of claim 3, wherein the dose of CBD ranges from about 5 mg/kg/day to about 25 mg/kg/day.

20. The method of claim 3, wherein the dose of CBD is about 10 mg/kg/day.

21. The method of claim 3, wherein the dose of CBD is about 20 mg/kg/day.

22. The method of claim 3, wherein the dose of CBD is about 25 mg/kg/day.
